(19) United States
(12) Patent Application Publication
(10) Pub. No.: US 2026/0354810 A1
(43) Pub. Date: Nov. 12, 2026

(54) ADJUSTABLE PENILE RETRACTION SYSTEM FOR UROLOGIC SURGERIES

(71) Applicants: James Scott Hay, Parkland, FL (US); Ashley Gonzalez, Clearwater, FL (US); Ross Simon, Wesley, FL (US)

(72) Inventors: James Scott Hay, Parkland, FL (US); Ashley Gonzalez, Clearwater, FL (US); Ross Simon, Wesley, FL (US)

(21) Appl. No.: 19/669,962

(22) Filed: May 6, 2026

Related U.S. Application Data
(60) Provisional application No. 63/800,795, filed on May 6, 2025.

(51) Int. Cl. A61B 17/02

(52) U.S. Cl. CPC A61B 17/0206

(57) ABSTRACT

The penile retraction system for use in a urologic surgical procedure includes a retractor body, two articulating fingers extending from the retractor body, a gripping mechanism at the distal ends of the articulating fingers, the gripping mechanism including structures that engage and stabilize a penis without excessive pressure, an interface operatively connected to the articulating fingers, the interface including concave structures configured to be manipulated by a user, wherein activation of the interface actuates the gripping mechanism to selectively engage and release the penis, a ratcheting system operatively connected to the articulating fingers, the ratcheting system including a gear-driven mechanism configured to permit movement of the articulating fingers in a first direction corresponding to engagement of the penis and resists movement in an opposite direction, and a surgical drape attachment element coupled with the retractor body, the surgical drape attachment element configured for securing the system to a surgical drape.

### CROSS-REFERENCE TO RELATED APPLICATIONS

This patent application claims priority to provisional patent application 63/800,795 filed on May 6, 2025. The subject matter of provisional patent application 63/800,795 is hereby incorporated by reference in its entirety.

### STATEMENT REGARDING FEDERALLY SPONSORED RESEARCH OR DEVELOPMENT

Not applicable.

### INCORPORATION BY REFERENCE OF MATERIAL SUBMITTED ON A COMPACT DISC

Not applicable.

### TECHNICAL FIELD

The claimed embodiments relate to the field of medical devices and more specifically to medical devices for penile retraction during urologic surgeries.

### BACKGROUND

Penile prosthesis implantation is a well-established surgical procedure used to treat erectile dysfunction that has not responded to other medical treatments. The procedure involves the implantation of a penile prosthetic into the patient for the purpose of restoring penile function. During the procedure, one or more incisions are made to allow for placement of the prosthetic components. The surgeon carefully dissects the necessary anatomical structures and inserts the prosthetic into the penile shaft, ensuring proper sizing and placement to achieve optimal function and cosmetic appearance.

A critical aspect of the procedure is maintaining the proper positioning of the penis throughout the surgery to allow for precise implantation of the prosthetic components. Traditionally, this role is fulfilled by an assisting individual who manually stabilizes and manipulates the penis to provide the surgeon with unobstructed access to the operative site. However, this manual approach presents several challenges. The necessity of continuous manual stabilization can lead to fatigue for the assisting individual, affecting consistency in positioning. Additionally, reliance on human assistance introduces variability, which can impact procedural efficiency and accuracy.

Moreover, maintaining sterility is a crucial concern in any surgical procedure, particularly in urological surgeries where the risk of infection is significant. The need for a secondary individual to hold the penis throughout the procedure increases the potential for inadvertent contamination, requiring meticulous adherence to sterile protocols. Furthermore, in procedures where a single surgeon operates without additional personnel, the absence of a dedicated assistant may complicate the process and prolong surgical time.

Despite advancements in surgical techniques and instrumentation, there remains a lack of standardized solutions to address the challenges associated with intraoperative penile stabilization. Existing techniques rely primarily on manual support, which introduces inconsistencies and potential inefficiencies. A need exists for improved methods or devices that can provide stable, reliable, and sterile support for penile positioning during inflatable penile prosthesis implantation while reducing reliance on manual assistance.

### SUMMARY

A penile retraction system for use in a urologic surgical procedure is provided. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claim subject matter's scope.

The penile retraction system for use in a urologic surgical procedure includes a retractor body configured to be positioned in relation to a surgical field, at least two articulating fingers extending from the retractor body, each articulating finger rotating about more than one fulcrum enabling multi-directional movement, a gripping mechanism at the distal ends of the at least two articulating fingers, the gripping mechanism including structures configured to engage and stabilize a penis without excessive pressure, an interface operatively connected to the at least two articulating fingers, the interface including concave structures configured to be manipulated by a user, wherein activation of the interface actuates the gripping mechanism to selectively engage and release the penis, a ratcheting system operatively connected to the at least two articulating fingers, the ratcheting system including a gear-driven mechanism configured to permit movement of the at least two articulating fingers in a first direction corresponding to engagement of the penis and resists movement in an opposite direction, and a surgical drape attachment element coupled with the retractor body, the surgical drape attachment element configured for securing the system to a surgical drape.

### BRIEF DESCRIPTION OF THE DRAWINGS

The accompanying drawings, which are incorporated in and constitute a part of this disclosure, illustrate various example embodiments. In the drawings:

FIG. 1 is an illustration of a rear view of the penile retraction system, according to an example embodiment;

FIG. 2 is an illustration of a right-side view of the penile retraction system, according to an example embodiment;

FIG. 3 is an illustration of a front view of the penile retraction system, according to an example embodiment;

FIG. 4 is an illustration of a left-side view of the penile retraction system, according to an example embodiment;

FIG. 5 is an illustration of a bottom view of the penile retraction system, according to an example embodiment;

FIG. 6 is an illustration of a top view of the penile retraction system, according to an example embodiment;

FIG. 7 is an illustration of an exploded view of the penile retraction system, according to an example embodiment;

FIG. 8 is an illustration of a perspective left side view of the penile retraction system, according to an example embodiment;

FIG. 9 is an illustration of a perspective right side view of the penile retraction system, according to an example embodiment;

FIG. 10 is an illustration of a perspective bottom view of the penile retraction system, according to an example embodiment.

FIG. 11 is a closeup illustration of a rear side view of the articulating fingers of the penile retraction system, according to an example embodiment.

### DETAILED DESCRIPTION

The following detailed description refers to the accompanying drawings. Wherever possible, the same reference numbers are used in the drawings and the following description to refer to the same or similar elements. While embodiments may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the claimed subject matter. Instead, the proper scope of the claimed subject matter is defined by the appended claims.

The claimed embodiments provides several advantages that address the drawbacks of current methods used during penile prosthesis implantation procedures. One of the primary benefits of the claimed embodiments is its ability to provide consistent and stable penile support throughout the surgical procedure. Unlike traditional manual stabilization, which can lead to variability due to human fatigue and positioning inconsistencies, the device ensures a fixed and reliable hold. This results in improved precision during implantation, reducing the need for frequent repositioning and minimizing procedural delays. Another key advantage is the enhanced sterility it offers. The traditional approach of having an assistant manually hold the penis increases the risk of inadvertent contamination, requiring meticulous adherence to sterile protocols. The claimed embodiments eliminate this concern by providing a hands-free retraction mechanism, reducing direct human contact with the surgical field. Additionally, the claimed embodiments significantly improve surgical efficiency by freeing up the assistant to focus on other critical aspects of the procedure. Manual stabilization requires continuous attention and can divert personnel resources away from other essential surgical tasks. By securing the penis in the optimal position without ongoing manual support, the claimed embodiments allow the surgical team to operate more efficiently, potentially reducing overall operative time.

Furthermore, the design of the claimed embodiments helps minimize soft tissue trauma, a common issue with stay-sutures and self-retaining retractors. Traditional methods of penile stabilization can exert excessive pressure on soft tissues, leading to postoperative pain, swelling, and complications. The claimed embodiments ensure a secure yet gentle hold, reducing tissue strain and improving patient recovery outcomes. Further, unlike rigid retraction systems that may not accommodate anatomical variations, the claimed embodiments feature adjustable components that conform to the patient's unique structure, enhancing both comfort and surgical effectiveness. Finally, by integrating modern material choices and mechanical features, the claimed embodiments ensure compatibility with intraoperative imaging techniques such as fluoroscopy, eliminating concerns about interference during critical surgical assessments.

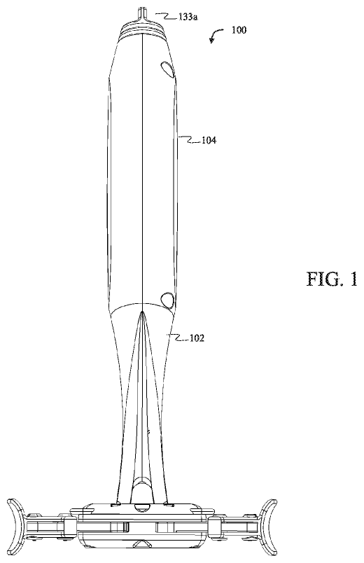

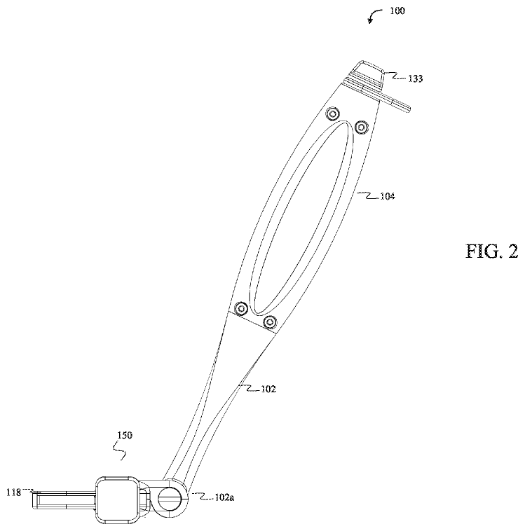

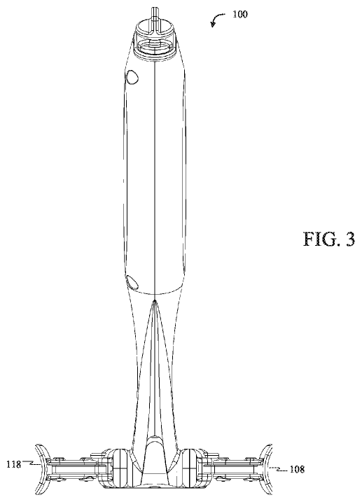

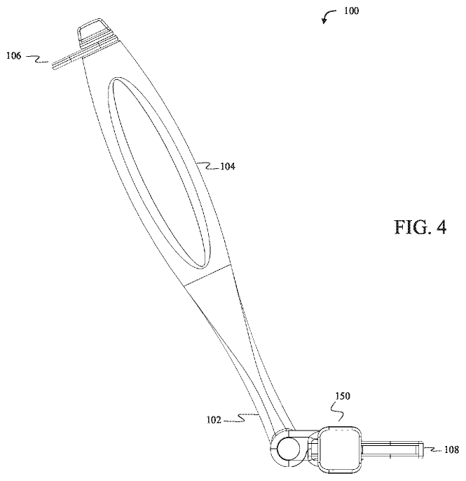

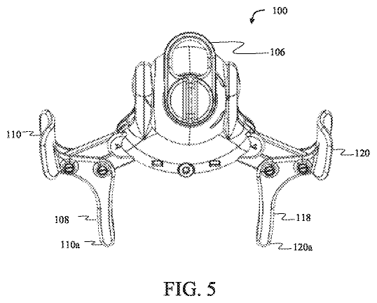

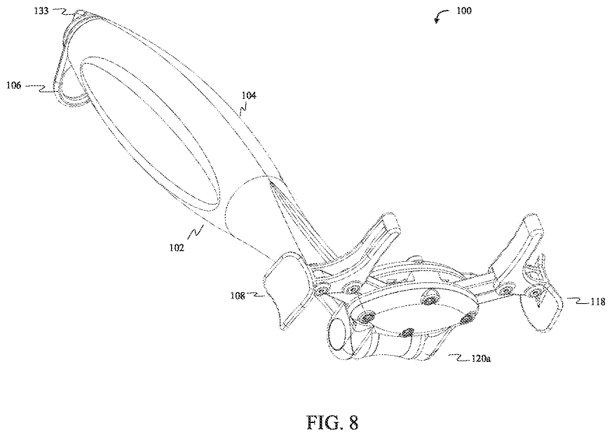

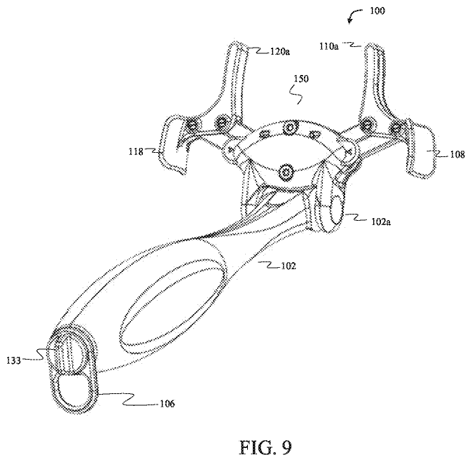

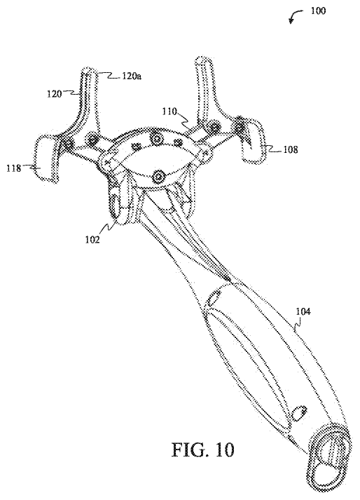

The claimed embodiments will now be described with reference to FIGS. 1-11. The penile retraction system 100 is designed to provide stable and hands-free retraction of the penis during urologic surgical procedures, particularly for penile prosthesis implantation. The system comprises a retractor body 102, which serves as the structural foundation of the device and is configured to be positioned relative to a surgical field. The retractor body 102 includes a handle 104, which allows for ease of positioning and manipulation by the surgeon or surgical assistant. At the base of the handle 104, a surgical drape attachment swivel ring 106 is provided, enabling secure attachment of the system to the surgical drape. The swivel ring 106 allows for minor adjustments in positioning without detaching from the drape, ensuring both stability and flexibility during the procedure.

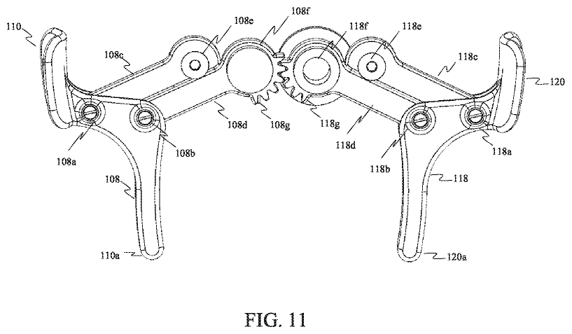

Extending from the retractor body 102 is a head assembly 150, which comprises the working end of the device 100. The head assembly comprises two articulating fingers, namely the first articulating finger 108 and the second articulating finger 118. These articulating fingers rotate about two fulcrums or hinges (described more fully below with reference to FIG. 11), allowing for multi-directional movement, and enabling the system to conform to various anatomical configurations.

Each articulating finger has two branches-one branch terminates in a concave-tipped end, while the other branch terminates in a gripping end. The branches extend in different directions and result in a rough V-shape. The first articulating finger 108 has a branch that terminates in the concave-tipped end 110 and the second articulating finger 118 has a branch that terminates in the concave-tipped end 120. These concave-tipped ends 110 and 120 are contoured and curved to allow for manipulation by the user's fingers or fingertips. The first articulating finger 108 has another branch that terminates in the gripping end 110a and the second articulating finger 118 has another branch that terminates in the gripping end 120a. These gripping ends 110a and 120a are contoured and curved to maximize engagement with the penis while distributing pressure evenly to minimize trauma to soft tissue. Activation of the concave-tipped ends 110, 120 results in controlled articulation of the fingers, allowing them to engage and stabilize the penis.

Each concave-tipped end 110, 120 may be a concave shaped oval configured to accept a finger or fingertip. Said oval may be placed roughly perpendicular to the branch on which it is located. The concave-tipped ends 110, 120 are configured for allowing the user, such as a surgeon or another healthcare worker, can easily grasp the concave-tipped ends 110, 120 using his thumb and forefinger. The user can pinch, push, pull or otherwise move or manipulate the concave-tipped ends 110, 120 using his thumb and forefinger, such that the fingers 108, 118 grasp or release a penis during a surgery or other medical procedure. This allows the user to easily make adjustments to the placement of the fingers 108, 118 and to enable them to grasp or release a penis during use.

The combination of the gripping ends 110a and 120a acts as a gripping mechanism configured to engage and stabilize a penis without excessive pressure, which is defined as any pressure above ~20-35 mmHg (≈2.7-4.7 kPa). The combination of the concave-tipped end 110 and the concave-tipped end 120 act as an interface operatively connected to the two articulating fingers, the interface configured to be manipulated by the user (which may be a surgeon or other healthcare worker), wherein activation of the interface actuates the gripping mechanism to selectively engage and release the penis. In certain embodiments, the gripping ends 110a and 120a are configured to distribute contact forces over a surface area sufficient to reduce localized pressure on soft tissue. The geometry of the gripping ends may include curved or contoured surfaces that conform to the anatomy of the penis, thereby enhancing engagement while minimizing tissue compression. In some embodiments, the gripping ends may include compliant materials or coatings that further distribute applied forces and reduce the likelihood of tissue trauma.

Clamp actuation and release are performed directly at the clamp interface operatively connected to the two articulating fingers via lateral finger pressure. This provides improved tactile feedback to the surgeon (or other healthcare worker), more precise incremental control and reduced mechanical complexity of the device. The clamp interface operatively connected to the two articulating fingers translates user input applied at the interface into corresponding movement of the articulating fingers. In certain embodiments, the transmission mechanism includes one or more pivoting linkages, gears, cams, or sliding elements configured to convert rotational or translational input at the interface into controlled articulation of the fingers. In some embodiments, the interface provides both actuation and control of the articulating fingers, including advancement toward an engaged position and release from the engaged position. The interface may be configured to provide tactile feedback to the user, such as resistance or incremental indexing, corresponding to movement of the articulating fingers.

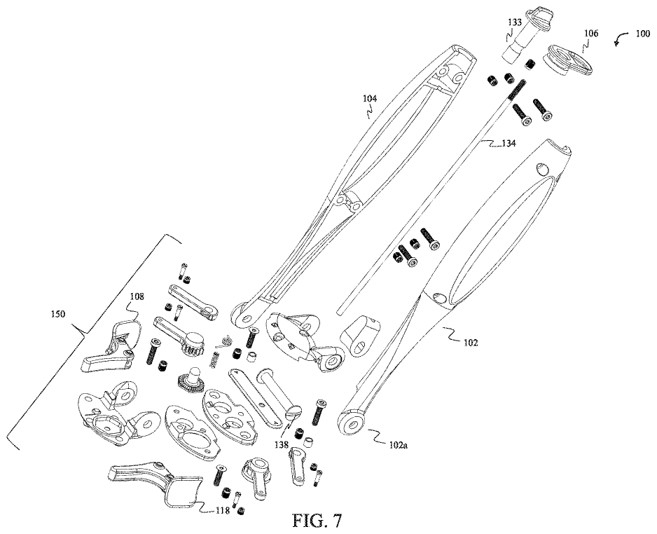

The fingers 108 and 118 may be coated with a thin silicone layer that has a slightly rough texture to enhance grip and minimize slippage, even in the presence of surgical fluids. The head assembly 150 is connected to the body 102 via hinge connection 102a, which may be held together with a pin or bolt 138 (see FIG. 7).

The movement control system is operatively connected to the articulating fingers 108 and 118, facilitating precise control over their movement and engagement with the penis. The movement control system includes the actuator 133 on the proximal end of the device 100, wherein the actuator is used to lock the articulating fingers 108 and 118 in place via a mechanical system. The actuator 133 may be a dial with a protruding flange 133a that extends upwards from the body of the dial, so as to provide a surface that may be manipulated by the user's hand and/or fingers. The actuator 133 is connected to a shaft 134, which extends through the retractor body 102 and is mechanically connected (via a series of connecting elements) to the first and second articulating fingers. Activation of the actuator 133 results in locking the movement of the fingers after engagement of the penis, so as to stabilize the penis. The actuator may be a circular input output device, a dial, a button, a switch, or the like. Thus, the movement control system comprises the actuator 133, the shaft 134, the articulating fingers 108 and 118 and all of the intervening parts that move together to result in the movement of the articulating fingers to engage and disengage the penis.

To ensure that the fingers 108 and 118 maintain their position once engagement is achieved, the system includes a locking mechanism which may be activated by pushing down, rotating, throttling, or otherwise interacting with it. The locking mechanism when engaged, prevents further movement of the articulating fingers, ensuring that the penis remains stabilized without unintentional displacement. The locking mechanism may be engaged by pressing down on the actuator 133.

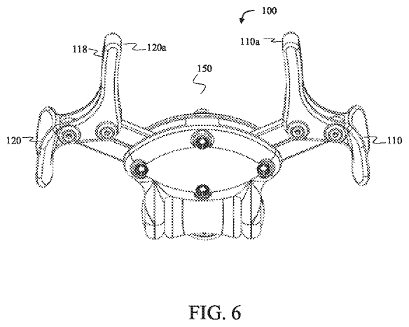

The exploded view of the penile retraction system 100, shown in FIG. 6, illustrates the individual components and their assembly within the device. The retractor body 102 is shown as the central structural component, with the handle 104 extending from it for ease of manipulation. The surgical drape attachment swivel ring 106 is depicted at the base of the handle 104, highlighting its function in securing the device to the surgical drape. The first articulating finger 108 and the second articulating finger 118 are displayed separately from the retractor body 102, revealing their joint construction. The concave-tipped ends 110 and 120 of the articulating fingers 108 and 118, respectively, are illustrated with their curved surfaces designed for engagement with the user's fingers or fingertips. The gripping ends 110a and 120a of the articulating fingers 108 and 118, respectively, are illustrated with their gripping surfaces designed for atraumatic engagement with the penis. The movement control system is depicted in an exploded manner, showing the actuator 133 and its connection to the shaft 134, which is in turn connected to the first and second articulating fingers 108, 118. The internal routing of the shaft is visible, demonstrating how its extends through the retractor body 102 to lock movement of the fingers 108 and 118. The exploded view provides a comprehensive representation of how each component interconnects, detailing the mechanical relationships that enable precise and stable penile retraction during surgical procedures.

The articulating fingers 108 and 118 utilize multiple hinges or joints, which allow for precise angulation in various configurations, such as circular, U-shaped, or tip-only adjustments. These hinges or joints allows for the guiding of movement and joint rotation. This design enables the surgeon to customize global finger articulation to accommodate different surgical requirements and anatomical variations. The fingers 108 and 118 further include a gripping ends (110a, 120a) that attach to the penis under the glans, providing secure stabilization while minimizing trauma.

To enhance surgical efficiency, the system features a quick-release mechanism that allows the articulating fingers 108 and 118 to rapidly disengage from the penis when necessary. The quick-release function is integrated into the movement control system and is actuated by pressing the actuator 133, which allows the fingers 108 and 118 to open quickly. This ensures that the device can be safely removed without causing unnecessary tissue damage.

FIG. 11 is a closeup illustration of a rear side view of the articulating fingers 108, 118 of the penile retraction system 100, according to an example embodiment. FIG. 11 shows that articulating arm 108 has two hinges about which is rotates or spins-hinge 108a and hinge 108b. First arm 108c is connected on one end to the first articulating arm 108 via hinge 108a and on the other end connected to the body of the head assembly via hinge 108e. Second arm 108d is connected on one end to the first articulating arm 108 via hinge 108b and on the other end connected to the body of the head assembly via hinge 108f. Note that a gear 108g with teeth is located around the hinge structure 108f.

FIG. 11 also shows that articulating arm 118 has two hinges or fulcrums about which is rotates or spins-hinge 118a and hinge 118b. First arm 118c is connected on one end to the articulating arm 118 via hinge 118a and on the other end connected to the body of the head assembly via hinge 118e. Second arm 118d is connected on one end to the articulating arm 118 via hinge 118b and on the other end connected to the body of the head assembly via hinge 118f. Note that a gear 118g with teeth is located around the hinge structure 118f.

As the user interacts with (i.e., moves) the concave-tipped features 110, 120, this interfaces with the hinges 108f and/or 118f to so as to rotate the hinges 108f and 118f. Since the teeth of the gears 108g and 118g mesh together, said hinges 108f and 118f rotate at the same pace. Rotation of the arms 108d and 118d about hinges 108f and 118f cause the articulating arms 108, 118 to rotate about hinges 108b, 118b, respectively. This movement, in turn, causes rotation of articulating arms 108, 118 about hinges 108a and 118a, which causes the arms 108c, 118c to rotate about hinges 108e, 118e, respectively. In this way, the double hinges about which each articulating arm 108, 118 rotates allows the articulating arms to move in various axes of freedom, so as to provide the user a large locus of space in which he may operate.

The meshing engagement between toothed members 108g and 118g ensures synchronized movement of the articulating fingers 108 and 118. This synchronization maintains symmetrical positioning and balanced force application during engagement of the penis. In contrast to independent finger movement systems, the synchronized configuration reduces the likelihood of uneven pressure distribution and enhances stability during surgical manipulation.

The arrangement of hinges 108a, 108b, 108e, 108f and corresponding hinges 118a, 118b, 118e, 118f defines a multi-link kinematic chain that enables compound articulation of the fingers 108, 118 across multiple axes. This configuration allows the articulating fingers to move along a controlled path that conforms to anatomical variations while maintaining alignment of the gripping ends relative to the penis. The use of multiple hinges provides increased degrees of freedom while maintaining mechanical stability during engagement.

The combination of the elements 108, 108c, 108d, 108g, 118, 118c, 118d, 118g act as a ratcheting system operatively connected to the two articulating fingers, the ratcheting system including a gear-driven mechanism configured to permit movement of the two articulating fingers in a first direction corresponding to engagement of the penis and resists movement in an opposite direction. The ratcheting system comprising one more toothed members (108g, 118g) and a pawl configured to engage the toothed members, wherein the pawl permits incremental movement of the two articulating fingers in a first direction corresponding to engagement of the penis and resists movement in an opposite direction absent user-actuated release of the pawl.

In certain embodiments, the ratcheting system further includes a pawl element biased into engagement with the toothed members (108g, 118g), such that rotation in a first direction corresponding to engagement is permitted, while reverse rotation is resisted unless the pawl is disengaged. The pawl may be disengaged via a user-actuated release mechanism integrated with the actuator (133) or another actuator. The ratcheting system provides incremental positional control of the articulating fingers, while the locking mechanism provides a static fixation state once a desired position is achieved. Both systems may share mechanical components while performing distinct functional roles.

In the illustrated embodiment of FIG. 11, the toothed members 108g and 118g are configured to cooperate with a pawl element (not shown) that is biased into engagement with the teeth of the toothed members. The pawl may be spring-biased or otherwise urged into engagement such that rotation of the toothed members in a first direction corresponding to engagement of the penis is permitted through incremental advancement, while rotation in an opposite direction is resisted. In this manner, the ratcheting system provides controlled, stepwise positioning of the articulating fingers 108, 118. Disengagement of the pawl from the toothed members may be achieved through actuation of a release mechanism operatively coupled to the interface 133, thereby allowing reverse movement of the articulating fingers when desired.

In certain embodiments, the toothed members 108g and 118g are mechanically coupled such that rotation of one toothed member results in synchronized rotation of the other, thereby ensuring coordinated movement of the articulating fingers 108 and 118. This synchronized movement maintains balanced engagement forces on opposing sides of the penis, improving stability and reducing localized pressure concentrations.

The retractor body 102 and its components may be constructed from a sterilizable, fluoroscopy-compatible polymer, making the device suitable for use in imaging-guided procedures without interference. The frame of the retractor body 102 may be strategically offset and positioned caudal to the surgical site, ensuring unobstructed access and optimal visualization of the operative field. This design prevents shadowing and interference with surgical lighting, allowing the surgeon to maintain a clear line of sight for precise dissection and implantation.

By securing the penis in an optimal position without requiring manual assistance, the penile retraction system 100 significantly improves procedural efficiency, reduces the risk of infection by minimizing direct skin contact, and eliminates the inconsistencies associated with manual stabilization. Additionally, the atraumatic design reduces the need for stay-sutures or invasive retraction methods, thereby decreasing postoperative pain and recovery time. The secure attachment of the device to the surgical drape via the surgical drape attachment swivel ring 106 ensures stability throughout the procedure, further enhancing its effectiveness as a surgical tool.

In one alternative embodiment, to ensure compatibility with intraoperative fluoroscopic imaging, the penile retraction system 100 may be constructed from a medical-grade plastic material. This plastic composition renders the device radiolucent, preventing interference with real-time imaging during the procedure. The radiolucent nature of the retractor body 102 allows surgeons to monitor implant placement with greater precision. The use of plastic also ensures the system is lightweight, making it easier to manipulate during surgery.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A penile retraction system for use in a urologic surgical procedure, comprising:
a) a retractor body configured to be positioned in relation to a surgical field;
b) at least two articulating fingers extending from the retractor body, each articulating finger rotating about more than one fulcrum enabling multi-directional movement;
c) a gripping mechanism at the distal ends of the at least two articulating fingers, the gripping mechanism including structures configured to engage and stabilize a penis without excessive pressure;
d) an interface operatively connected to the at least two articulating fingers, the interface including concave structures configured to be manipulated by a user, wherein activation of the interface actuates the gripping mechanism to selectively engage and release the penis;
e) a ratcheting system operatively connected to the at least two articulating fingers, the ratcheting system including a gear-driven mechanism configured to permit movement of the at least two articulating fingers in a first direction corresponding to engagement of the penis and resists movement in an opposite direction; and
f) a surgical drape attachment element coupled with the retractor body, the surgical drape attachment element configured for securing the system to a surgical drape.

2. The penile retraction system of claim 1, wherein the at least two articulating fingers are coated with a silicone layer having a textured surface.

3. The penile retraction system of claim 1, further comprising a locking mechanism configured to secure the at least two articulating fingers in a fixed position once engagement of the penis is achieved.

4. The penile retraction system of claim 1, wherein the ratcheting system further comprises a quick-release mechanism configured for rapid disengagement of the articulating fingers from the penis when actuated.

5. The penile retraction system of claim 1, wherein the concave structures of the interface are contoured to accommodate fingertips of the user.

6. The penile retraction system of claim 1, wherein the surgical drape attachment element includes a swivel ring, the swivel ring allowing for minor adjustments to the retractor body's positioning without detaching from the drape.

7. The penile retraction system of claim 1, wherein the at least two articulating fingers are made from a flexible, sterilizable polymer.

8. The penile retraction system of claim 1, wherein each of the at least two articulating fingers is connected to a pair of arms via said more than one fulcrum.

9. The penile retraction system of claim 1, wherein the interface provides tactile feedback to the user.

10. The penile retraction system of claim 1, wherein the retractor body is configured to be radiolucent.

11. A penile retraction system for use in a urologic surgical procedure, comprising:
a) a retractor body configured to be positioned in relation to a surgical field;
b) at least two articulating fingers extending from the retractor body, each articulating finger rotating about more than one fulcrum enabling multi-directional movement;
c) a gripping mechanism at the distal ends of the at least two articulating fingers, the gripping mechanism including structures configured to engage and stabilize a penis without excessive pressure;
d) an interface operatively connected to the at least two articulating fingers, the interface including concave structures configured to be manipulated by a user, wherein activation of the interface actuates the gripping mechanism to selectively engage and release the penis;
e) a ratcheting system operatively connected to the at least two articulating fingers, the ratcheting system including a gear-driven mechanism configured to permit movement of the at least two articulating fingers in a first direction corresponding to engagement of the penis and resists movement in an opposite direction;
f) a locking mechanism configured to secure the at least two articulating fingers in a fixed position once engagement of the penis is achieved; and
g) a surgical drape attachment element coupled with the retractor body, the surgical drape attachment element configured for securing the system to a surgical drape.

12. The penile retraction system of claim 11, wherein the at least two articulating fingers are coated with a silicone layer having a textured surface.

13. The penile retraction system of claim 11, wherein the more than one fulcrum comprises two hinges.

14. The penile retraction system of claim 11, wherein the ratcheting system further comprises a quick-release mechanism configured for rapid disengagement of the articulating fingers from the penis when actuated.

15. The penile retraction system of claim 11, wherein the concave structures of the interface are contoured to accommodate fingertips of the user.

16. The penile retraction system of claim 11, wherein the surgical drape attachment element includes a swivel ring, the swivel ring allowing for minor adjustments to the retractor body's positioning without detaching from the drape.

17. The penile retraction system of claim 11, wherein the at least two articulating fingers are made from a flexible, sterilizable polymer.

18. The penile retraction system of claim 11, wherein each of the at least two articulating fingers is connected to a pair of arms via said more than one fulcrum.

19. The penile retraction system of claim 11, wherein the interface provides tactile feedback to the user.

20. The penile retraction system of claim 11, wherein the retractor body is configured to be radiolucent.
